SEQUENCE LISTING (12 sequences)

SEQ ID NO: 1 - RNA, 72 nt, Homo sapiens
ugucggguagcuuaucagacugauguugacuguugaaucucauggcaacaccagucgaugggcugucugaca

SEQ ID NO: 2 - RNA, 73 nt, Homo sapiens
ggccggcugggguuccuggggaugggauuugcuuccugucacaaaucacauugccagggauuuccaaccgacc

SEQ ID NO: 3 - RNA, 97 nt, Homo sapiens
cucaggugcucuggcugcuuggguuccuggcaugcugauuugugacuuaagauuaaaaucacauugccagggauuaccacgcaaccacgaccuuggc

SEQ ID NO: 4 - RNA, 22 nt, Homo sapiens
uagcuuaucagacugauguuga

SEQ ID NO: 5 - RNA, 21 nt, Homo sapiens
caacaccagucgaugggcugu

SEQ ID NO: 6 - RNA, 22 nt, Homo sapiens
gggguuccuggggaugggauuu

SEQ ID NO: 7 - RNA, 21 nt, Homo sapiens
aucacauugccagggauuucc

SEQ ID NO: 8 - RNA, 22 nt, Homo sapiens
uggguuccuggcaugcugauuu

SEQ ID NO: 9 - RNA, 21 nt, Homo sapiens
aucacauugccagggauuacc

SEQ ID NO: 10 - RNA, 88 nt, Homo sapiens
aucucuuacacaggcugaccgauuucuccugguguucagagucuguuuuugucuagcaccauuugaaaucgguuaugauguaggggga

SEQ ID NO: 11 - RNA, 22 nt, Homo sapiens
ugaccgauuucuccugguguuc

SEQ ID NO: 12 - RNA, 22 nt, Homo sapiens
uagcaccauuugaaaucgguua

(19) United States
(12) Patent Application Publication
(10) Pub. No.: US 2018/0010194 A1
(43) Pub. Date: Jan. 11, 2018

(54) Methods for the Diagnosis of Pancreatic Cancer

(71) Applicants: INSERM (Institut National de la Sante et de la Recherche Medicale), Paris (FR); Universite Paul Sabatier Toulouse III, Toulouse (FR); Centre Hospitalier Universitaire de Toulouse, Toulouse (FR)

(72) Inventors: Pierre Cordelier, Toulouse Cedex 1 (FR); Louis Buscail, Toulouse Cedex 1 (FR); Marine Humeau, Toulouse Cedex 1 (FR)

(21) Appl. No.: 15/542,801

(22) PCT Filed: Jan. 12, 2016
(86) PCT No.: PCT/EP2016/050418

§ 371 Date: Jul. 11, 2017

(30) Foreign Application Priority Data
Jan. 12, 2015 (EP) 15305020.8

(51) Int. Cl. C12Q 1/68

(52) U.S. Cl. CPC C12Q 1/6886; C12Q 2600/178; C12Q 2600/118; C12Q 2600/158

(57) ABSTRACT

The present invention relates to the diagnosis of pancreatic cancer, in particular to a salivary mi RNA for use in the diagnosis of pancreatic cancer.

### FIELD OF THE INVENTION

The present invention relates to the diagnosis of pancreatic cancer.

### BACKGROUND OF THE INVENTION

Pancreatic ductal adenocarcinoma (PDAC, pancreatic cancer) is the fourth leading cause of cancer death in Western countries, with the lowest five-year relative (1) and 1-year survival (2) rates among commonly diagnosed cancers. Pancreatic cancer is anticipated to move to the second leading cause of cancer death worldwide by 2020 in the absence of improvements in treatment (3). There are currently no means for the reliable diagnosis of early stages of pancreatic cancer. Consequently, the vast majority of patients (85%) display an advanced disease that results in a low resection rate leading to a dismal overall median survival of 4 to 6 months.

Accordingly, there is an urgent need to develop means for early diagnosis of pancreatic cancer to allow curative surgery. Thus, the discovery of early biomarkers for pancreatic cancer diagnosis is highly desirable and will favor early patient management and prognosis.

MicroRNAs (miRNAs) have recently emerged as a new class of robust biomarkers for cancer diagnosis, including pancreatic cancer (4). These potent regulators of gene expression can be thoroughly quantified in diverse tissues and fluids, due to their inherent high stability as compared to proteins and messenger RNAs. Of importance, miRNAs can be quantified in very low amounts of material, including micro-biopsies, and in highly degraded samples. Recent reports extensively demonstrated that miRNA profiles in biopsies can successfully discriminate normal from cancerous pancreatic tissue, and may also predict cancer prognosis or response to treatment (4). The stability of miRNAs has been once again underscored as miRNA profiling in plasma was recently demonstrated to differentiate pancreatic cancer patients from healthy controls (4). Such findings pave the way for the use of circulating miRNAs as minimally-invasive pancreatic cancer biomarkers, to prevent from unnecessary biopsies.

Several other body fluids such as urine, semen and saliva have been recently considered as repositories for cancer diagnosis (5, 6). Saliva has the superior advantage as sample collection is simple, non-invasive, causes little anxiety on the part of patients and can be repeated. Saliva has been demonstrated to contain proteins/peptides, nucleic acids, electrolytes, and hormones that originate from both local and systemic sources and recent studies have prompted interest in using saliva as a source of biomarkers. Accordingly, the use of saliva for detection of oral diseases has been extensively demonstrated (7), and saliva recently emerged as a wealthy source of miRNAs, such as has-miR-31, for oral cancer diagnosis (8-11). On the other hand, saliva use for systemic disease is largely unclear.

There is no disclosure in the art of robust biomarkers and the use of salivary miRNAs for early pancreatic cancer diagnosis.

### SUMMARY OF THE INVENTION

The present invention relates to the diagnosis of pancreatic cancer.

Particularly, the present invention relates to a method of identifying a subject having or at risk of having or developing pancreatic cancer, comprising a step of measuring in a saliva sample obtained from said subject the expression level of at least one miRNA selected from the group consisting of miR-21, miR-23a, miR-23b and miR-29c.

### DETAILED DESCRIPTION OF THE INVENTION

Salivary miRNAs expression profile in pancreatic cancer was investigated by inventors using saliva samples from patients with pancreatic cancer, precancerous lesions (Intraductal papillary mucinous neoplasms (IPMNs)), inflammatory disease (pancreatitis), and cancer-free patients. The inventors also investigated the kinetic of salivary miRNA detection in experimental model of pancreatic cancer. The inventors found that hsa-miR-21, hsa-miR-23a, hsa-miR-23b and hsa-miR-29c are significantly deregulated in saliva of pancreatic cancer patients compared to control and successfully segregate pancreatic cancer patients from cancer-free donors. Interestingly, the inventors also demonstrated that miR-23a and miR23b are overexpressed in the saliva of patients with pancreatic cancer precursor lesions. The inventors also demonstrated that salivary miRNA detection precedes tumour burden in an experimental model of pancreatic cancer.

The present invention establishes a reliable endogenous control of miRNAs for salivary-based diagnostic, shows significant differences in miRNA profiles between saliva from patients with pancreatic cancer and saliva from patients that are tumour-free and indicates that the salivary miRNA profiles are discriminatory in pancreatic cancer patients. The present invention stems for the use of salivary miRNAs as early biomarkers for non-invasive pancreatic cancer diagnosis.

### Diagnostics Methods

Accordingly, the present invention relates to a method of identifying a subject having or at risk of having or developing pancreatic cancer, comprising a step of measuring in a saliva sample obtained from said subject the expression level of at least one miRNA selected from the group consisting of miR-21, miR-23a and miR-23b.

In a further aspect, the method of the invention comprises the step of measuring in a saliva sample obtained from the subject the expression level of at least one miRNA selected from the group consisting of miR-21, miR-23a, miR-23b and miR-29c.

Typically, 1, 2, 3, or 4 miRNAs selected from the group consisting of miR-21, miR-23a, miR-23b and miR-29c are measured.

As used herein, the term “miR” has its general meaning in the art and refers to the miRNA sequence publicly available from the data base http://microrna.sanger.ac.uk/sequences/ under the miRBase Accession number. The miRNAs of the invention are listed in Table A:

TABLE A
list of the miRNAs according to the invention

| miRNA | miRBase Accession number |
|---|---|
| miRNA-21 | MI0000077 |
| miRNA-23a | MI0000079 |
| miRNA-23b | MI0000439 |
| miRNA-21-5p | MIMAT0000076 |
| miRNA-21-3p | MIMAT0004494 |
| miRNA-23a-5p | MIMAT0004496 |
| miRNA-23a-3p | MIMAT0000078 |
| miRNA-23b-5p | MIMAT0004587 |
| miRNA-23b-3p | MIMAT0000418 |
| miRNA-29c | MI0000735 |
| hsa-miR-29c-5p | MIMAT0004673 |
| hsa-miR-29c-3p | MIMAT0000681 |

As used herein, the term “subject” denotes a mammal. In a preferred embodiment of the invention, a subject according to the invention refers to any subject (preferably human) afflicted with or susceptible to be afflicted with pancreatic cancer. In one embodiment of the invention, a subject according to the invention refers to any subject (preferably human) afflicted with intraductal papillary mucinous neoplasms (IPMNs), a clinical situation at risk of developing pancreatic cancer.

The term “pancreatic cancer” has its general meaning in the art and also refers to Pancreatic ductal adenocarcinoma (PDAC) (1-4, 15).

The term “Intraductal Papillary Mucinous Neoplasms” or “IPMNs” has its general meaning in the art and refers to non-invasive precursor lesions of pancreatic cancer (15). The term “Intraductal Papillary Mucinous Neoplasms” or “IPMNs” also relates to pancreatic cancer precursor lesions.

As used herein, the term “saliva sample” refers to saliva sample derived from the subject that contains nucleic acid materials. Said saliva sample is obtained for the purpose of the in vitro evaluation.

A further aspect of the invention relates to a method of identifying a subject having or at risk of having or developing pancreatic cancer, comprising a step of measuring in a saliva sample obtained from said subject the expression level of miR-21 and miR-23a.

A further aspect of the invention relates to a method of identifying a subject having or at risk of having or developing pancreatic cancer, comprising a step of measuring in a saliva sample obtained from said subject the expression level of miR-21 and miR-23b.

A further aspect of the invention relates to a method of identifying a subject having or at risk of having or developing pancreatic cancer, comprising a step of measuring in a saliva sample obtained from said subject the expression level of miR-23a and miR-23b.

A further aspect of the invention relates to a method of identifying a subject having or at risk of having or developing pancreatic cancer, comprising a step of measuring in a saliva sample obtained from said subject the expression level of all miRNAs of the group consisting of miR-21, miR-23a and miR-23b.

A further aspect of the invention relates to a method of identifying a subject having or at risk of having or developing pancreatic cancer, comprising a step of measuring in a saliva sample obtained from said subject the expression level of miR-21 and miR-29c.

A further aspect of the invention relates to a method of identifying a subject having or at risk of having or developing pancreatic cancer, comprising a step of measuring in a saliva sample obtained from said subject the expression level of miR-23a and miR-29c.

A further aspect of the invention relates to a method of identifying a subject having or at risk of having or developing pancreatic cancer, comprising a step of measuring in a saliva sample obtained from said subject the expression level of miR-23b and miR-29c.

A further aspect of the invention relates to a method of identifying a subject having or at risk of having or developing pancreatic cancer, comprising a step of measuring in a saliva sample obtained from said subject the expression level of miR-21, miR-23a and miR-29c.

A further aspect of the invention relates to a method of identifying a subject having or at risk of having or developing pancreatic cancer, comprising a step of measuring in a saliva sample obtained from said subject the expression level of miR-21, miR-23b and miR-29c.

A further aspect of the invention relates to a method of identifying a subject having or at risk of having or developing pancreatic cancer, comprising a step of measuring in a saliva sample obtained from said subject the expression level of miR-23a, miR-23b and miR-29c.

A further aspect of the invention relates to a method of identifying a subject having or at risk of having or developing pancreatic cancer, comprising a step of measuring in a saliva sample obtained from said subject the expression level of all miRNAs of the group consisting of miR-21, miR-23a, miR-23b and miR-29c.

The method of the invention may further comprise a step consisting of comparing the expression level of at least one miRNA in the saliva sample with a reference value, wherein detecting differential in the expression level of the miRNA between the saliva sample and the reference value is indicative of subject having or at risk of having or developing a pancreatic cancer.

In one embodiment, the reference value may correspond to the expression level determined in a saliva sample associated with a healthy subject not afflicted with pancreatic cancer. Accordingly, a higher expression level of at least one miRNA selected from the group consisting of miR-21, miR-23a, miR-23b and miR-29c than the reference value is indicative of a subject having or at risk of having or developing a pancreatic cancer, and a lower or equal expression level of at least one miRNA selected from the group consisting of miR-21, miR-23a, miR-23b and miR-29c than the reference value is indicative of a subject not having or not at risk of having or developing a pancreatic cancer.

In another embodiment, the reference value may correspond to the expression level determined in a saliva sample associated with a subject afflicted with pancreatic cancer. Accordingly, a higher or equal expression level of at least one miRNA selected from the group consisting of miR-21, miR-23a, miR-23b and miR-29c than the reference value is indicative of a subject having or at risk of having or developing a pancreatic cancer, and a lower expression level of at least one miRNA selected from the group consisting of miR-21, miR-23a, miR-23b and miR-29c than the reference value is indicative of a subject not having or not at risk of having or developing a pancreatic cancer.

According to the invention, measuring the expression level of the miRNA selected from the group consisting of miR-21, miR-23a, miR-23b and miR-29c of the invention in the saliva sample obtained from the subject can be performed by a variety of techniques.

For example the nucleic acid contained in the samples (saliva prepared from the subject) is first extracted according to standard methods, for example using lytic enzymes or chemical solutions or extracted by nucleic-acid-binding resins following the manufacturer's instructions. Conventional methods and reagents for isolating RNA from a saliva sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

The expression level of one or more miRNA in the saliva sample may be determined by any suitable method. Any reliable method for measuring the level or amount of miRNA in a sample may be used. Generally, miRNA can be detected and quantified from a saliva sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

In some embodiments, RNA is converted to DNA (cDNA) prior to analysis. cDNA can be generated by reverse transcription of isolated miRNA using conventional techniques. miRNA reverse transcription kits are known and commercially available. Examples of suitable kits include, but are not limited to the mirVana TaqMan® miRNA transcription kit (Ambion, Austin, Tex.), and the TaqMan® miRNA transcription kit (Applied Biosystems, Foster City, Calif.). Universal primers, or specific primers, including miRNA-specific stem-loop primers, are known and commercially available, for example, from Applied Biosystems. In some embodiments, miRNA is amplified prior to measurement. In some embodiments, the expression level of miRNA is measured during the amplification process. In some embodiments, the expression level of miRNA is not amplified prior to measurement. Some exemplary methods suitable for determining the expression level of miRNA in a sample are described in greater hereinafter. These methods are provided by way of illustration only, and it will be apparent to a skilled person that other suitable methods may likewise be used.

Many amplification-based methods exist for detecting the expression level of miRNA nucleic acid sequences, including, but not limited to, PCR, RT-PCR, qPCR, and rolling circle amplification. Other amplification-based techniques include, for example, ligase chain reaction (LCR), multiplex ligatable probe amplification, in vitro transcription (IVT), strand displacement amplification (SDA), transcription-mediated amplification (TMA), nucleic acid sequence based amplification (NASBA), RNA (Eberwine) amplification, and other methods that are known to persons skilled in the art. A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a miRNA) may be performed prior to PCR amplification. Reverse transcription reactions include the use of, e.g., a RNA-based DNA polymerase (reverse transcriptase) and a primer. Kits for quantitative real time PCR of miRNA are known, and are commercially available. Examples of suitable kits include, but are not limited to, the TaqMan® miRNA Assay (Applied Biosystems) and the mirVana™ qRT-PCR miRNA detection kit (Ambion). The miRNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence. In some embodiments, custom qRT-PCR assays can be developed for determination of miRNA levels. Custom qRT-PCR assays to measure miRNAs in a sample can be developed using, for example, methods that involve an extended reverse transcription primer and locked nucleic acid modified PCR. Custom miRNA assays can be tested by running the assay on a dilution series of chemically synthesized miRNA corresponding to the target sequence. This permits determination of the limit of detection and linear range of quantitation of each assay. Furthermore, when used as a standard curve, these data permit an estimate of the absolute abundance of miRNAs measured in the samples. Amplification curves may optionally be checked to verify that Ct values are assessed in the linear range of each amplification plot. Typically, the linear range spans several orders of magnitude. For each candidate miRNA assayed, a chemically synthesized version of the miRNA can be obtained and analyzed in a dilution series to determine the limit of sensitivity of the assay, and the linear range of quantitation. Relative expression levels may be determined, for example, according to the 2(−ΔΔ C(T)) Method, as described by Livak et ah, Analysis of relative gene expression data using real-time quantitative PCR and the 2(−ΔΔ C(T)) Method. Methods (2001) December; 25(4):402-8.

In some embodiments, two or more miRNAs are amplified in a single reaction volume. For example, multiplex q-PCR, such as qRT-PCR, enables simultaneous amplification and quantification of at least two miRNAs of interest in one reaction volume by using more than one pair of primers and/or more than one probe. The primer pairs comprise at least one amplification primer that specifically binds each miRNA, and the probes are labeled such that they are distinguishable from one another, thus allowing simultaneous quantification of multiple miRNAs.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al, Am. J. Pathol. (2001) 159(0:63-69; Nallur et al, Nucleic Acids Res. (2001) 29(23):E118). In the presence of two primers, one hybridizing to the (+) strand of DNA, and the other hybridizing to the (−) strand, a complex pattern of strand displacement results in the generation of over 10⁹ copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of miRNA sequence and expression level at very low miRNA concentrations (see, for example, Cheng et al., Angew Chem. Int. Ed. Engl. (2009) 48(18):3268-72; Neubacher et al, Chembiochem. (2009) 10(8): 1289-91).

miRNAs quantification method may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values.

Many miRNA quantification assays are commercially available from Qiagen (S. A. Courtaboeuf, France), Exiqon (Vedbaek, Denmark) or Applied Biosystems (Foster City, USA).

Expression level of miRNAs may be expressed as absolute expression level or normalized expression level. Typically, expression levels are normalized by correcting the absolute expression level of miRNAs by comparing its expression to the expression of a mRNA that is not a relevant for determining subject having or at risk of having or developing a pancreatic cancer, e.g., a housekeeping mRNA that is constitutively expressed. Suitable mRNA for normalization include housekeeping mRNAs such as the U6, U24, U48 and S18. This normalization allows the comparison of the expression level in one sample, e.g., a subject sample, to another sample, or between samples from different sources. In a particular embodiment, expression levels are normalized by correcting the absolute expression level of miRNAs by comparing its expression to the expression of a reference miRNA such as miR-92a.

Nucleic acids exhibiting sequence complementarity or homology to the miRNAs of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

The probes and primers are “specific” to the miRNAs they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

miRNA may be detected using hybridization-based methods, including but not limited to hybridization arrays (e.g., microarrays), NanoString analysis, Northern Blot analysis, branched DNA (bDNA) signal amplification, and in situ hybridization.

Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, inkjet printing, or electrochemistry on microelectrode arrays. Also useful are microfluidic TaqMan Low-Density Arrays, which are based on an array of microfluidic qRT-PCR reactions, as well as related microfluidic qRT-PCR based methods. In one example of microarray detection, various oligonucleotides (e.g., 200+ 5′-amino-modified-C6 oligos) corresponding to human sense miRNA sequences are spotted on three-dimensional CodeLink slides (GE Health/Amersham Biosciences) at a final concentration of about 20 μMand processed according to manufacturer's recommendations. First strand cDNA synthesized from 20 μg TRIzol-purified total RNA is labeled with biotinylated ddUTP using the Enzo BioArray end labeling kit (Enzo Life Sciences Inc.). Hybridization, staining, and washing can be performed according to a modified Affymetrix Antisense genome array protocol. Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each miRNA. Each miRNA signal can be transformed to log base 2, and a one-sample t test can be conducted. Independent hybridizations for each sample can be performed on chips with each miRNA spotted multiple times to increase the robustness of the data.

Microarrays can be used for the expression profiling of miRNAs. For example, RNA can be extracted from the sample and, optionally, the miRNAs are size-selected from total RNA. Oligonucleotide linkers can be attached to the 5′ and 3′ ends of the miRNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the, capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA. Total RNA containing the miRNA extracted from the sample can also be used directly without size-selection of the miRNAs. For example, the RNA can be 3′ end labeled using T4 RNA ligase and a fluorophore-labeled short RNA linker. Fluorophore-labeled miRNAs complementary to the corresponding miRNA capture probe sequences on the array hybridize, via base pairing, to the spot at which the capture probes are affixed. The fluorescence intensity of each spot is then evaluated in terms of the number of copies of a particular miRNA, using a number of positive and negative controls and array data normalization methods, which will result in assessment of the level of expression of a particular miRNA. Several types of microarrays can be employed including, but not limited to, spotted oligonucleotide microarrays, pre-fabricated oligonucleotide microarrays or spotted long oligonucleotide arrays.

Accordingly, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

RT-PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase. The majority of the thermocyclers on the market now offer similar characteristics. Typically, thermocyclers involve a format of glass capillaries, plastics tubes, 96-well plates or 384-wells plates. The thermocylcer also involve a software analysis.

miRNAs can also be detected without amplification using the nCounter Analysis System (NanoString Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter miRNA assay kits are available from NanoString Technologies, which are capable of distinguishing between highly similar miRNAs with great specificity. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Mass spectroscopy can be used to quantify miRNA using RNase mapping. Isolated RNAs can be enzymatically digested with RNA endonucleases (RNases) having high specificity (e.g., RNase Tl, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESTMS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside. Matrix-assisted laser desorption/ionization mass spectrometry (MALDI-MS) has also been used as an analytical approach for obtaining information about posttranscriptionally modified nucleosides. MALDI-based approaches can be differentiated from ESTbased approaches by the separation step. In MALDI-MS, the mass spectrometer is used to separate the miRNA. To analyze a limited quantity of intact miRNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR® XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact miRNAs are eluted from the trap column and directly injected into a CI 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for miRNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple miRNAs can be detected in a microarray format using a novel approach that combines a surface enzyme reaction with nanoparticle-amplified SPR imaging (SPRI). The surface reaction of poly(A) polymerase creates poly(A) tails on miRNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for miRNA profiling at attamole levels. miRNAs can also be detected using branched DNA (bDNA) signal amplification (see, for example, Urdea, Nature Biotechnology (1994), 12:926-928). miRNA assays based on bDNA signal amplification are commercially available. One such assay is the QuantiGene® 2.0 miRNA Assay (Affymetrix, Santa Clara, Calif.). Northern Blot and in situ hybridization may also be used to detect miRNAs. Suitable methods for performing Northern Blot and in situ hybridization are known in the art. Advanced sequencing methods can likewise be used as available. For example, miRNAs can be detected using Illumina® Next Generation Sequencing (e.g. Sequencing-By-Synthesis or TruSeq methods, using, for example, the HiSeq, HiScan, GenomeAnalyzer, or MiSeq systems (Illumina, Inc., San Diego, Calif.)). miRNAs can also be detected using Ion Torrent Sequencing (Ion Torrent Systems, Inc., Gulliford, Conn.), or other suitable methods of semiconductor sequencing.

In one embodiment, the present invention relates to a method of identifying a subject having or at risk of having or developing pancreatic cancer, comprising the steps of:

i) providing a saliva sample from a subject,

ii) measuring the expression level of at least one miRNA selected from the group consisting of miR-21, miR-23a and miR-23b in the saliva sample obtained at step i),

iii) comparing said expression level measured in step ii) with a reference value, wherein detecting differential in said expression level between the saliva sample and the reference value is indicative of a subject having or at risk of having or developing a pancreatic cancer.

In a further aspect, the method of identifying a subject having or at risk of having or developing pancreatic cancer of the invention comprises the step of measuring in a saliva sample obtained from the subject the expression level of at least one miRNA selected from the group consisting of miR-21, miR-23a, miR-23b and miR-29c.

A further aspect of the invention relates to a method of preventing or treating pancreatic cancer in a subject in need thereof comprising the steps of:

i) providing a saliva sample from a subject,

ii) measuring the expression level of at least one miRNA selected from the group consisting of miR-21, miR-23a and miR-23b in the saliva sample obtained at step i),

iii) comparing said expression level measured in step ii) with a reference value, wherein detecting differential in said expression level between the saliva sample and the reference value is indicative of a subject having or at risk of having or developing a pancreatic cancer, and

iv) treating said subject having or at risk of having or developing a pancreatic cancer with a pancreatic cancer treatment.

In a further aspect, the method of preventing or treating pancreatic cancer of the invention comprises the step of measuring in a saliva sample obtained from the subject the expression level of at least one miRNA selected from the group consisting of miR-21, miR-23a, miR-23b and miR-29c.

A further aspect of the invention relates to a method for monitoring the efficacy of a treatment for a pancreatic cancer in a subject in need thereof.

Methods of the invention may be applied for monitoring the treatment (e.g., drug compounds) of the subject. For example, the effectiveness of an agent to affect the expression level of the miRNAs according to the invention may be monitored during treatments of subjects receiving pancreatic cancer treatments.

The term “pancreatic cancer treatment” refers to any type of pancreatic cancer therapy undergone by the pancreatic cancer subjects including surgical resection of pancreatic cancer, gemcitabine, fluorouracil, FOLFIRINOX (fluorouracil, irinotecan, oxaliplatin, and leucovorin), nab-paclitaxel, inhibitors of programmed death 1 (PD-1), PD-1 ligand PD-L1, anti-CLA4 antibodies, EGFR inhibitors such as erlotinib, chemoradiotherapy, inhibitors of PARP, inhibitors of Sonic Hedgehog, gene therapy and radiotherapy.

Accordingly, the present invention relates to a method for monitoring the treatment of a subject affected with a pancreatic cancer, said method comprising the steps consisting of:

i) diagnosis of pancreatic cancer before said treatment by performing the method of the invention,

ii) assessing the states of pancreatic cancer after said treatment by performing the method of the invention,

iii) and comparing the results determined a step i) with the results determined at step ii) wherein a difference between said results is indicative of the effectiveness of the treatment.

### Kits

The invention also relates to kits for performing the methods of the invention, wherein said kits comprise means for measuring the expression level of the miRNA of the invention in the saliva sample obtained from the subject. The kits may include probes, primers, macroarrays or microarrays as above described.

For example, the kit may comprise a set of miRNA probes as above defined, usually made of DNA, and optionally pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards.

Alternatively the kit of the invention may comprise amplification primers (e.g. stem-loop primers) that may be pre-labelled or may contain an affinity purification or attachment moiety. The kit may further comprise amplification reagents and also other suitably packaged reagents and materials needed for the particular amplification protocol.

In a particular embodiment, the invention relates to a kit for identifying a subject having or at risk of having or developing pancreatic cancer, comprising means for measuring, in a saliva sample obtained from said subject, the expression level of at least one miRNA selected from the group consisting of miR-21, miR-23a and miR-23b.

In a further aspect, the kit of the invention comprises means for measuring, in a saliva sample obtained from said subject, the expression level of at least one miRNA selected from the group consisting of miR-21, miR-23a, miR-23b and miR-29c.

In a particular embodiment, the invention relates to a kit for identifying a subject having or at risk of having or developing pancreatic cancer, comprising means for measuring, in a saliva sample obtained from said subject, the expression level of miR-21 and miR-23a, miR-2 land miR-23b, miR-23a and miR-23b, miR-21 and miR-29c, miR-23a and miR-29c, or miR-23b and miR-29c.

In a particular embodiment, the invention relates to a kit for identifying a subject having or at risk of having or developing pancreatic cancer, comprising means for measuring, in a saliva sample obtained from said subject, the expression level of miR-21, miR-23a and miR-23b, the expression level of miR-21, miR-23a and miR-29c, the expression level of miR-23a, miR-23b and miR-23b, or the expression level of miR-21, miR-23a, miR-23b and miR-29c.

In a particular embodiment, the kit of the invention relates to a kit which further comprises means for comparing the expression level of the miRNA in the saliva sample with a reference value, wherein detecting differential in the expression level of the miRNA between the saliva sample and the reference value is indicative of a subject having or at risk of having or developing a pancreatic cancer.

Oligonucleotide sequences:

>SEQ ID NO: 1 for hsa-miR-21 MI0000077 (pre-miRNA)
UGUCGGGUAGCUUAUCAGACUGAUGUUGACUGUUGAAUCUCAUGGCAA
CACCAGUCGAUGGGCUGUCUGACA
>SEQ ID NO: 2 for hsa-miR-23a MI0000079 (pre-
miRNA)
GGCCGGCUGGGGUUCCUGGGGAUGGGAUUUGCUUCCUGUCACAAAUCAC
AUUGCCAGGGAUUUCCAACCGACC
>SEQ ID NO: 3 for hsa-miR-23b MI0000439 (pre-
miRNA)
CUCAGGUGCUCUGGCUGCUUGGGUUCCUGGCAUGCUGAUUUGUGACUUA
AGAUUAAAAUCACAUUGCCAGGGAUUACCACGCAACCACGACCUUGGC
>SEQ ID NO: 4 for hsa-miR-21-5p MIMAT0000076
(mature miRNA)
UAGCUUAUCAGACUGAUGUUGA
>SEQ ID NO: 5 for hsa-miR-21-3p MIMAT0004494
(mature miRNA)
CAACACCAGUCGAUGGGCUGU
>SEQ ID NO: 6 for hsa-miR-23a-5p MIMAT0004496
(mature miRNA)
GGGGUUCCUGGGGAUGGGAUUU
>SEQ ID NO: 7 for hsa-miR-23a-3p MIMAT0000078
(mature miRNA)
AUCACAUUGCCAGGGAUUUCC
>SEQ ID NO: 8 for hsa-miR-23b-5p MIMAT0004587
(mature miRNA)
UGGGUUCCUGGCAUGCUGAUUU
>SEQ ID NO: 9 for hsa-miR-23b-3p MIMAT0000418
(mature miRNA)
AUCACAUUGCCAGGGAUUACC
>SEQ ID NO: 10 for hsa-miR-29c MI0000735 (pre-
miRNA)
AUCUCUUACACAGGCUGACCGAUUUCUCCUGGUGUUCAGAGUCUGUUUU
UGUCUAGCACCAUUUGAAAUCGGUUAUGAUGUAGGGGGA
>SEQ ID NO: 11 for hsa-miR-29c-5p MIMAT0004673
(mature miRNA)
UGACCGAUUUCUCCUGGUGUUC
>SEQ ID NO: 12 for hsa-miR-29c-3p MIMAT0000681
(mature miRNA)
UAGCACCAUUUGAAAUCGGUUA

The invention will be further illustrated by the following figures and examples. However, these examples and figures should not be interpreted in any way as limiting the scope of the present invention.

### FIGURES

FIG. 1: Salivary miRNA profiles in pancreatic cancer patients.

Analysis of miRNA profiles in saliva samples from patients with pancreatic cancer (n=7) or cancer-free patients (n=4). Results are mean±S.D. of ΔCq: (Cq of miRNA of interest−Cq of miR-92a) done in triplicate. *, P<0.05

FIG. 2: Salivary miRNA profiles in pancreatic cancer and pancreatitis patients.

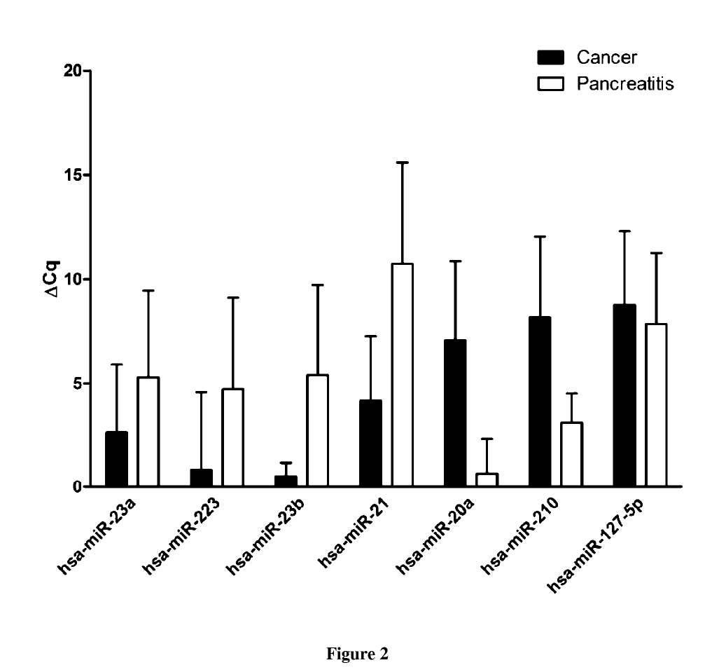

Analysis of miRNA profiles in saliva samples from patients with pancreatic cancer (n=7) or pancreatitis (n=4). Results are mean±S.D. of ΔCq: (Cq of miRNA of interest−Cq of miR-92a) done in triplicate.

FIG. 3: Salivary miRNA profiles in an experimental model of pancreatic cancer.

Analysis of salivary hsa-miR-21, hsa-miR-23a, hsa-miR-23b levels and Lucia blood levels in mice xenografted with Mia PACA-2 Lucia cells at the time indicated following tumour induction. Results are mean±S.D. of 6 biological replicates done in experimental triplicates. miRNA levels are expressed in Cq, Lucia levels are expressed in relative light units (r.l.u.). The grey zone corresponds to tumour burden monitoring using secreted Lucia.

### EXAMPLES

### Example 1

Material & Methods

Patients

This protocol was approved by the Ethical Committee (Comité de Protection des Personnes Sud-Ouest et Outre Mer N^{o} 1, number 1-10-21). To avoid blood contamination, patients were asked not to brush their teeth within 45 minutes prior to sample collection. Saliva was collected using sterile tips and micropipettes during endoscopic examination under general anesthesia with propofol. Saliva was immediately placed in pre-chilled 1.5-ml microcentrifuge tubes containing and equal volume of Saliva protect reagent (Qiagen) and stored at −80° C. until ready for use. In the present invention, the inventors included patients aged >18 years who had given their written informed consent. Other criteria for inclusion were no contraindications for general anesthesia or for endoscopic ultrasound. Fine needle aspiration material was used for histological, cytological and molecular (KRAS activating mutation analysis (12)) diagnosis of pancreatitis or pancreatic cancer. Twenty-one patients were included in this study; 7 were diagnosed with pancreatic cancer, 4 were diagnosed with pancreatitis (either acute or chronic) and 4 had unrelated digestive diseases (control group) (Table 1). Patients with no diagnosis (n=2), diagnosed with other digestive cancers (n=2) or intraductal papillary mucinous neoplasia (n=2) were excluded from the study.

TABLE 1
Patients' characteristics

| | | | |
|---|---|---|---|
| Patient # | Age | Diagnostic | KRAS status |

Group: Control

| | | |
|---|---|---|
| 13 | 64 | colon polyps |
| 14 | 81 | Gallstones |
| 15 | 70 | colon polyps |
| 16 | 66 | hypochondriac |
| mean | 70 | |
| | (64-81) | |

Group: Pancreatitis

| | | |
|---|---|---|
| 4 | 54 | Chronic pancreatitis |
| 17 | 39 | Acute pancreatits |
| 18 | 51 | Acute pancreatits |
| 19 | 54 | Chronic pancreatitis |
| mean | 50 | |
| | (39-54) | |

Group: Cancer

| | | | |
|---|---|---|---|
| 3 | 59 | Pancreatic adenocarcinoma | positive |
| 6 | 66 | Pancreatic adenocarcinoma | positive |
| 7 | 66 | Pancreatic adenocarcinoma | negative |
| 8 | 68 | Pancreatic adenocarcinoma | positive |
| 9 | 68 | Pancreatic adenocarcinoma | positive |
| 10 | 67 | Pancreatic adenocarcinoma | positive |
| 12 | 74 | Pancreatic adenocarcinoma | positive |
| mean | 66 | | |
| | (59-74) | | |

### Experimental Protocol

All animals experiments were conducted according to the national ethical guidelines for experimental research and protocol were approved by the regional ethical committee for animal experimentation and were performed in accordance with the Guide for the Care and Use of Laboratory Animals (US National Institutes of Health). Human pancreatic cancer-derived Mia PACA-2 cells expressing secreted Lucia luciferase (13, 14) are grown in RPMI medium supplemented with 10% fetal calf serum, L-glutamine, an antibiotic, an antimycotic cocktail (Life Technologies), and Plasmocin® (InvivoGen) in a humidified incubator at 37° C. in 5% CO₂. Six two-week-old female nu/nu mice were anesthetized by intraperitoneal injection of pentobarbital (80 mg/kg) diluted in 0.9% NaCl, supplemented with oral anaesthesia using oxygen/isofluorane (2.5 mixture) and Mia PACA-2 Lucia cells were implanted in the tail of pancreas as previously described (13, 14). Saliva secretion was not stimulated by pilocarpine. Saliva was obtained from the oral cavity by micropipette and immediately placed in pre-chilled 1.5-ml microcentrifuge tubes containing and equal volume of Saliva protect reagent (Qiagen). Collection was completed in 20 minutes and samples were stored at −80° C. until analyzed. For non-invasive tracking of tumour growth, blood was sampled by retro-orbital collection and centrifuged at 1000×g for 10 min in microcentrifuge tubes treated with EDTA. Lucia production was measured in 5 μl of plasma using coelenterazine (50 μM) as a substrate. For miRNA quantification studies, tumours were frozen in liquid nitrogen and stored at −80° C. until use.

RNA Extraction

Before saliva samples were used, they were defrosted on ice and centrifuged for 15 minutes at 2600×g at 4° C. The cell free supernatant was collected from the pellet and used immediately in the next step. Total RNA was isolated from 250 μL saliva supernatant and from tumours using Trizol LS reagent (Life technologies) and miRNAeasy extraction kit (Qiagen), respectively. DNase I treatment (DNase I, Qiagen) was used to remove contaminating DNA during RNA extraction. The concentration of total RNA was measured using Nanodrop N-100.

miRNA Quantification

Total salivary, cellular or tumour RNA (20 ng) was reverse transcribed and pre-amplified using the Universal cDNA synthesis kit (Exiqon), followed by Specific Target Amplification (STA) using TaqMan® PreAmp Master Mix (Life technologies) and pooled 94 microRNA LNA™ PCR primer sets (Exiqon). Following 15 pre-amplification cycles, STA reactions were diluted 1:10 in nuclease free water. qPCR Assay Mix consisted of TaqMan® Gene Expression Master Mix (Life technologies), DNA Binding Dye Sample Loading Reagent (Fluidigm), EvaGreen (Biorad), Forward and Reverse primer mix (Exiqon) and Assay Loading Reagent, and prepared as per the manufacturer's recommendations. Samples and sample mix was loaded on a Fluidigm chip (Fluidgm) and quantitative real time PCR reaction was run at 95° C. for 10 minutes, followed by 30 cycles at 95° C. for 10 seconds and 60° C. for 1 minute on the Fluidigm platform (Fluidgm). The quantification cycle (Cq) value is defined as the cycle number in the fluorescence emission, which exceeds that of a fixed threshold. A Cq of 15 to 30 was considered high expression and a Cq of 35 is considered low expression. A Cq value more than 40 was considered as undetectable miRNA. For miRNA quantitative PCR (qPCR) experiments, has-miR-92a was used as the reference gene in human saliva samples. The inventors calculated ΔCq by subtracting the Cq value of the reference miRNA (has-miR-92a) from the Cq value of each candidate miRNA biomarker. Data normalization was conducted using RQ manager 1.2.1 and Data Assist v3.0 from Applied Biosystems.

Statistical Analysis

The qPCR-based gene expression values between the different groups were compared using the nonparametric Wilcoxon rank-sum test. Candidate biomarker miRNAs were then selected based on P<0.05.

Results

Identification of Pancreatic Cancer-Specific Salivary miRNAs

In the present invention, 94 miRNAs were selected from the literature as follow: previously reported biomarkers for cancer, previously reported biomarkers for pancreatic cancer, detected in blood of patients with cancer or detected in saliva of patients with cancer. Expression of candidate miRNAs was screened by q(RT)PCR using Biomark Fluidgm in patients with pancreatic cancer (n=7), benign pancreatitis (n=4) or without cancer (n=4).

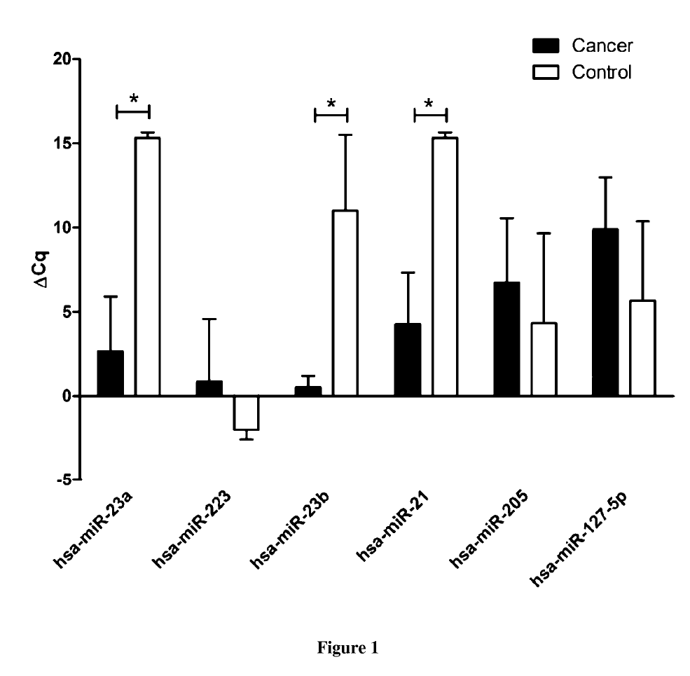

Of the 94 miRNAs, 23 miRNAs were undetectable in all samples tested. On the other hand, hsa-miR-23a, hsa-miR-223, hsa-miR-23b, hsa-miR-92a, hsa-miR-21, hsa-miR-205 and hsa-miR-127-5p were expressed at high levels in the saliva of pancreatic cancer and control patients. Using Genorm software, the inventors selected hsa-miR-92a as reference miRNA for this study. The inventors found that 3 miRNAs (has-miR-21, has-miR23a and has-miR-23b) were differently expressed in saliva from patients with pancreatic cancer (n=7) compared with control patients (n=4; Wilcoxon test, P<0.05) (Table 2 and FIG. 1). The expression of candidate miRNAs was higher (mean 3844 fold) in saliva samples from patients with tumours compared with expression in saliva samples from cancer-free patients (Table 2). Of importance, hsa-miR-21 and hsa-miR-23a were strictly specific for pancreatic cancer (100%) with excellent sensitivity (71.4% and 85.7%, respectively). hsa-miR-20a and hsa-miR-210 showed a trend as discriminatory miRNAs between pancreatic cancer and benign pancreatitis patients (0.09>P>0.05) (Table 3). Taken together, the invention demonstrates that salivary hsa-miR-21, hsa-miR-23a and hsa-miR-23b are novel non-invasive biomarkers for pancreatic cancer diagnosis.

TABLE 2
Average ΔCq values.

| | Cancer | | Control | | | | |
|---|---|---|---|---|---|---|---|
| | mean | | mean | | | | |
| | ΔCq | SD | ΔCq | SD | p | ΔΔCq | fold increase |
| hsa-miR-23a | 2.00 | 3.00 | 15.00 | 0.33 | 0.011 | −13.00 | 8192 |
| hsa-miR-223 | 0.86 | 3.00 | −2.00 | 0.58 | 0.293 | | |
| hsa-miR-23b | 0.54 | 0.65 | 11.00 | 4.00 | 0.002 | −10.46 | 1411 |
| hsa-miR-21 | 4.00 | 3.00 | 15.00 | 0.33 | 0.022 | −11.00 | 2048 |
| hsa-miR-205 | 6.00 | 3.00 | 4.00 | 5.00 | 0.36 | | |
| hsa-miR- | 9.00 | 3.00 | 5.00 | 4.00 | 0.23 | | |
| 127-5p | | | | | | | |

The expression of miRNAs in whole saliva from patients with pancreatic cancer (n=7) were compared to the expression of miRNAs in whole saliva from patients without cancer (n=4).

TABLE 3
Average ΔCq values.

| | Cancer | | Pancreatitis | | |
|---|---|---|---|---|---|
| | mean ΔCq | SD | mean ΔCq | SD | p |
| hsa-miR-23a | 2.00 | 3.00 | 5.00 | 4.00 | 0.341 |
| hsa-miR-223 | 0.86 | 3.00 | 4.00 | 4.00 | 0.293 |
| hsa-miR-23b | 0.54 | 0.65 | 5.00 | 4.00 | 0.154 |
| hsa-miR-21 | 4.17 | 3.00 | 10.73 | 4.00 | 0.164 |
| hsa-miR-20a | 7.00 | 3.00 | 0.68 | 1.00 | 0.128 |
| hsa-miR-210 | 8.00 | 3.00 | 3.00 | 1.00 | 0.186 |
| hsa-miR-127-5p | 8.00 | 3.00 | 7.00 | 3.00 | 0.440 |

The expression of miRNAs in whole saliva from patients with pancreatic cancer (n=7) were compared to the expression of miRNAs in whole saliva from patients with pancreatitis (n=4).

Salivary miRNAs Precede Tumour Burden in Experimental Models of Pancreatic Cancer

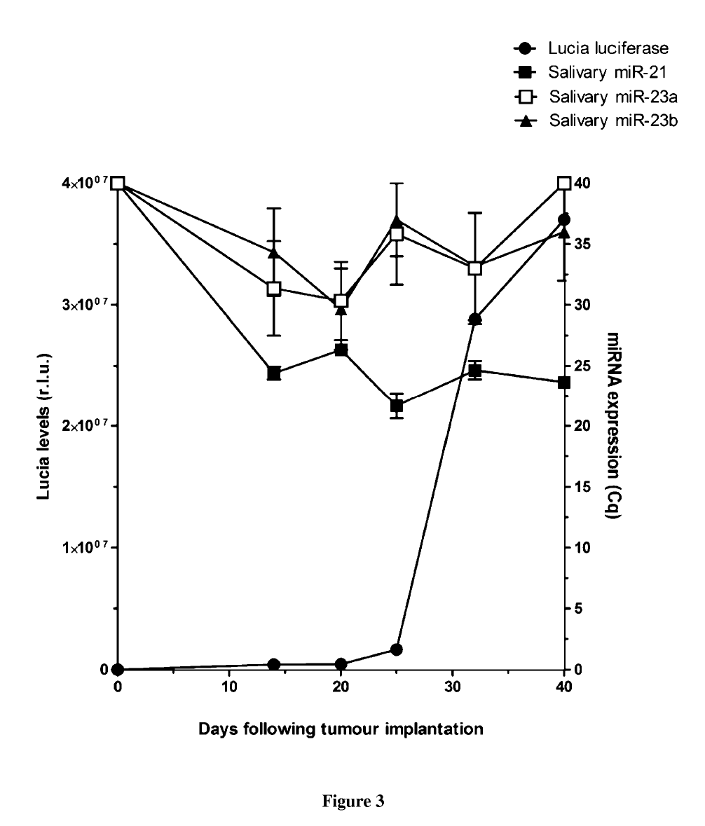

The inventors next investigated the kinetic of salivary miRNA detection in experimental model of pancreatic cancer. Mia PACA-2 human-derived pancreatic cancer cells were implanted in the pancreas of athymic mice (n=6). The inventors found that these cells and resulting xenografts express high levels of hsa-miR-21, hsa-miR-23a, hsa-miR-23b and has-miR-29c. As these cells were engineered to express secreted luciferase for non-invasive tumour monitoring (13, 14), pancreatic cancer tumours were detected 25 days following tumour cell engraftment and before they became palpable (FIG. 3).

Interestingly, hsa-miR-21 was readily detected in saliva from tumour-bearing mice, as soon as 14 days following tumour induction (FIG. 3), while undetectable in the saliva of tumour-free animals. In addition, salivary hsa-miR-21 expression remained elevated during the course of the experiment (FIG. 3). On the other hand, salivary hsa-miR-23a, hsa-miR-23b and has-miR-29c were detected at low levels (FIG. 3). Thus, the inventors validate hsa-miR-21 has a salivary biomarker in this experimental model of pancreatic cancer; in addition our results strongly demonstrate that salivary miRNA can be used for the early diagnosis of pancreatic cancer.

### Example 2

The inventors explored in the present invention the differences in salivary microRNA profiles between patients with pancreatic tumors that are not eligible for surgery (unrespectable pancreatic cancer), precancerous lesions (Intraductal papillary mucinous neoplasms (IPMNs)), inflammatory disease (pancreatitis) or cancer-free patients as an early diagnostic tool.

In addition to the patients described in the table 1, the inventors included in the study patients diagnosed with intraductal papillary mucinous neoplasia (IPMN) (n=2) (Table 4).

TABLE 4
Patients' characteristics
Group: Benign pancreatic masses

| Patient # | Age | Diagnostic |
|---|---|---|
| 21 | 52 | IPMN (secondary branch ducts) |
| 22 | 83 | IPMN (mixed) |

Results

In the present invention, 94 miRNAs were selected. Expression of candidate miRNAs was screened by q(RT)PCR using Biomark Fluidgm in patients with pancreatic cancer (n=7), pancreatitis (n=4), intraductal papillary mucinous neoplasia (IPMN, n=2) or without cancer (n=4) (Table 1 and Table 4).

Of the 94 miRNAs, 23 miRNAs were undetectable in all samples tested. The inventors found that 4 miRNAs (hsa-miR-21, hsa-miR23a, hsa-miR-23b and hsa-miR-29c) were significantly expressed in saliva from patients with pancreatic cancer (n=7), while undetectable in the saliva of control patients (n=4; Wilcoxon test, 0.001<p<0.03) (Table 5). The expression of the candidate miRNAs was strictly specific of pancreatic cancer (100%) with excellent sensitivity (ranging from 57% to 86%, Table 5). The has-miR-23a and hsa-miR-23b were detected in the saliva of patients diagnosed with IPMN, a well characterized precursor lesion of PDAC.

TABLE 5
Average Cq values, sensitivity and specificity
of the candidate microRNAs.

| | Cancer | | Control | | | | |
|---|---|---|---|---|---|---|---|
| | mean | | mean | | | | |
| | Cq | SD | Cq | SD | p | specificity | Sensitivity |
| hsa-miR-21 | 28.00 | 3.10 | 40.00 | 0.00 | 0.012 | 100% | 71% |
| hsa-miR-23a | 24.90 | 2.63 | 40.00 | 0.00 | 0.001 | 100% | 86% |
| hsa-miR-23b | 25.97 | 2.55 | 36.75 | 3.25 | 0.014 | 100% | 86% |
| hsa-miR-29c | 31.76 | 2.92 | 40.00 | 0.00 | 0.03 | 100% | 57% |

The expression of miRNAs in whole saliva from patients with PDAC (n=7) were compared to the expression of miRNAs in whole saliva from patients without cancer (n=4). The p value (nonparametric Wilcoxon rank-sum test) is indicated.

### REFERENCES

Throughout this application, various references describe the state of the art to which this invention pertains. The disclosures of these references are hereby incorporated by reference into the present disclosure.
1. Cancer Facts & FIGS. 2013 [Internet]. [cited 2014 Feb. 10]; Available from: http://www.cancer.org/research/cancerfactsfigures/cancerfactsfigures/cancer-facts-figures-2013
2. Burris H A 3rd, Moore M J, Andersen J, Green M R, Rothenberg M L, Modiano M R, Cripps M C, Portenoy R K, Storniolo A M, Tarassoff P, Nelson R, Don F A, Stephens C D, Von Hoff D D. Improvements in survival and clinical benefit with gemcitabine as first-line therapy for patients with advanced pancreas cancer: a randomized trial. J Clin Oncol Off J Am Soc Clin Oncol 1997; 15:2403-13 [PMID: 9196156]
3. Pancreatic Cancer Action Network [Internet]. Available from: http://www.pancan.org/4.
4. Humeau M, Torrisani J, Cordelier P. miRNA in clinical practice: Pancreatic cancer. Clin Biochem 2013; [PMID: 23570860 DOI: 10.1016/j.clinbiochem.2013.03.019]
5. Xiao Y-F, Yong X, Fan Y-H, Lü M-H, Yang S-M, Hu C-J. microRNA detection in feces, sputum, pleural effusion and urine: novel tools for cancer screening (Review). Oncol Rep 2013; 30:535-44 [PMID: 23754129 DOI: 10.3892/or.2013.2525]
6. Allegra A, Alonci A, Campo S, Penna G, Petrungaro A, Gerace D, Musolino C. Circulating microRNAs: new biomarkers in diagnosis, prognosis and treatment of cancer (review). Int J Oncol 2012; 41:1897-912 [PMID: 23026890 DOI: 10.3892/ijo.2012.1647]
7. Cheng Y-S L, Rees T, Wright J. A review of research on salivary biomarkers for oral cancer detection. Clin Transl Med 2014; 3:3 [PMID: 24564868 DOI: 10.1186/2001-1326-3-3]
8. Yoshizawa J M, Wong D T W. Salivary microRNAs and oral cancer detection. Methods Mol Biol Clifton N.J. 2013; 936:313-24 [PMID: 23007518 DOI: 10.1007/978-1-62703-083-0_24]
9. Matse J H, Yoshizawa J, Wang X, Elashoff D, Bolscher J G M, Veerman E C I, Bloemena E, Wong D T W. Discovery and prevalidation of salivary extracellular microRNA biomarkers panel for the noninvasive detection of benign and malignant parotid gland tumors. Clin Cancer Res Off J Am Assoc Cancer Res 2013; 19:3032-8 [PMID: 23575476 DOI: 10.1158/1078-0432.CCR-12-3505]
10. Xie Z, Chen G, Zhang X, Li D, Huang J, Yang C, Zhang P, Qin Y, Duan Y, Gong B, Li Z. Salivary microRNAs as promising biomarkers for detection of esophageal cancer. PloS One 2013; 8:e57502 [PMID: 23560033 DOI: 10.1371/journal.pone.0057502]
11. Brinkmann O, Wong D T W. Salivary transcriptome biomarkers in oral squamous cell cancer detection. Adv Clin Chem 2011; 55:21-34 [PMID: 22126022]
12. Bourret B, Selves J, Grand D, Danjoux M, Hanoun N, Cordelier P, Buscail L. Endoscopic Ultrasound-guided Fine-Needle Aspiration Biopsy Coupled With a KRAS Mutation Assay Using Allelic Discrimination Improves the Diagnosis of Pancreatic Cancer. J Clin Gastroenterol 2014; [PMID: 24798941 DOI: 10.1097/MCG.0000000000000053]
13. Delpu Y, Lulka H, Sicard F, Saint-Laurent N, Lopez F, Hanoun N, Buscail L, Cordelier P, Torrisani J. The Rescue of miR-148a Expression in Pancreatic Cancer: An Inappropriate Therapeutic Tool. PLoS ONE 2013; 8:e55513 [DOI: 10.1371/journal.pone.0055513]
14. Sicard F, Gayral M, Lulka H, Buscail L, Cordelier P. Targeting miR-21 for the Therapy of Pancreatic Cancer. Mol Ther [Internet] 2013 [cited 2013 Mar. 18]; [DOI: 10.1038/mt.2013.35] Available from: http://www.nature.com/doifinder/10.1038/mt.2013.35
15. Ryan DP1, Hong T S, Bardeesy N. Pancreatic adenocarcinoma. N Engl J Med. 2014 Sep. 11; 371(11):1039-49 [doi: 10.1056/NEJMra1404198].

1. A method of identifying a subject having or at risk of having or developing pancreatic cancer, comprising a step of measuring in a saliva sample obtained from said subject an expression level of at least one miRNA selected from the group consisting of miR-21, miR-23a and miR-23b.

2. The method according to claim 1 further comprising a step of comparing the expression level of the at least one miRNA in the saliva sample with a reference value, wherein detecting a differential between the expression level of the miRNA in the saliva sample and the reference value is indicative of the subject having or being at risk of having or developing a pancreatic cancer.

3. A method of preventing or treating pancreatic cancer in a subject in need thereof comprising the steps of:
i) identifying a subject having or at risk of having or developing pancreatic cancer by performing the method according to claim 1, and
ii) treating said subject having or at risk of having or developing a pancreatic cancer with a pancreatic cancer treatment.

4. A method for monitoring treatment of a subject affected with a pancreatic cancer comprising the steps of:
i) diagnosing pancreatic cancer before said treatment by performing the method according to claim 1,
ii) assessing the status of the pancreatic cancer after said treatment by performing the method according to claim 1, and
iii) comparing the results determined at step with the results determined at step ii) wherein a difference between the results determined at step i) and the results determined at step ii) is indicative of the effectiveness of the treatment.
